(19) United States
(12) Patent Application Publication
(10) Pub. No.: US 2003/0216789 A1
(43) Pub. Date: Nov. 20, 2003

(54) Method and system for treating sleep apnea

(75) Inventors: Mark E. Deem, Woodside, CA (US); Ron French, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE FOUNDRY, INC., Redwood City, CA

(21) Appl. No.: 10/431,077

(22) Filed: May 6, 2003

Related U.S. Application Data
(60) Provisional application No. 60/380,657, filed on May 14, 2002.

(51) Int. Cl. A61N 1/36

(52) U.S. Cl. 607/9

(57) ABSTRACT

Systems and apparatus for treating obstructive sleep apnea comprise an external generator and an implantable stimulator. The implantable stimulator includes an electrode which is placed in a target muscle or nerve which when stimulated will alleviate the symptoms of sleep apnea. The generator produces a radiofrequency or microwave signal which is broadcast to an antenna within the implanted stimulator. The implanted stimulator produces a stimulatory output, preferably without any other energy source.

### CROSS-REFERENCES TO RELATED APPLICATIONS

The present application is a non-provisional of U.S. Patent Application Serial No. 60/380,657 (Attorney Docket No. 020979-001200US), filed May 14, 2002, the full disclosure of which is incorporated herein by reference.

### BACKGROUND OF THE INVENTION

1. Field of the Invention

The present invention relates generally to medical apparatus and methods. More particularly, the present invention relates to methods and apparatus for alleviating sleep apnea.

Sleep apnea is a condition characterized by the temporary but reoccurring suspension of breathing during sleep. The condition affects those who are overweight, who have obstructions in their upper airways, or who have a neurological disorder. In those who have airway obstructions, the disease is generally referred to as “obstructive sleep apnea.” Sleep apnea can be a very serious condition in some patients, and a number of treatment approaches have been evolved over the years.

Of particular interest to the present invention, it has been proposed to treat sleep apnea by electrically stimulating certain muscles or nerves associated with a patient's breathing. For example, implantable electronic stimulators for stimulating the diaphragm, the upper airway muscles, the genioglossus, and the like. It has also bee proposed to overpace the heart in order to alleviate the symptoms of sleep apnea. Usually, these pacing systems are combined with sensors which detect the onset of apnea in a variety of ways. For example, the sensors may monitor minute ventilation, blood oxygen, respiration rate, intrathoracic pressure, diaphragm muscle stress, EEG waves, chest wall excursions, patient movement, pulse oximetry, or the like. Recent scientific publications have supported the conclusion that atrial pacing can reduce the occurrence of sleep apnea.

Systems intended to treat sleep apnea have generally relied on a fully implantable pacing or stimulatory device. The implanted device would include a sensor to detect an apnea condition and electrode or other stimulatory component for driving a target muscle or nerve. Such fully implantable systems have several drawbacks. First, they may require a relatively traumatic operation for implantation. Second, it can be difficult to reprogram or modify the operational characteristics of the implanted device. Third, the devices will usually require battery replacement or frequent recharging in order to continue working. Fourth, the ability to monitor certain feedback parameters, such as EEG or pulse oximetry, may be difficult to incorporate into a fully implantable system. For all these reasons, prior treatment approaches have been suitable only for those patients most seriously at risk from sleep apnea.

For these reasons, it would be desirable to provide improved systems and methods for treating and alleviating sleep apnea in patients. In particular, the systems and methods should allow for implantation of all implantable components via minimally invasive and/or endovascular approaches. The implantable components(s) should be very small and few in number, both to facilitate implantation and to reduce the risk of failure. The systems should permit simple reprogramming to allow for optimized treatment of the patients. The systems should further be easy to disable or turn off during those periods where the patient is awake or for any other reason wishes to refrain from using the system. The systems should be easy to use while the patient is asleep, and would preferably provide for both continuous treatment during the patient's sleep and/or selective treatment at only those times when the system determines that an apnea condition exists. Additionally, it would be desirable if the implantable component(s) of the system were to be unpowered, i.e., receive all power from an external source which broadcasts the power and treatment parameters to the implantable component(s). At least some of these objectives will be met by the invention described hereinafter.

2. Description of the Background Art

Systems and methods for treating sleep apnea are described in U.S. Pat. Nos. 6,269,269; 6,251,126; 6,126,611; 5,591,216; 5,540,733; 5,522,862; 5,483,969; 5,549,655; 5,485,851; 5,546,952; 5,122,354; 5,185,080; 5,146,918; 5,215,087; 5,174,287; 5,211,173; 5,300,094; 5,233,983; 5,946,680; 6,132,384; 6,240,310; 6,345,200; 5,335,657; U.S.01/0010010; U.S.01/0018547 and WO 00/01438. Implantable pacing and nerve stimulators which can operate from broadcast power and/or signals are described in U.S. Pat. Nos. 5,766,228; 4,543,955; 4,166,470; 4,134,408; 3,942,535; and 3,773,051. U.S. Pat. No. 3,773,051 describes a system for stimulating the phrenic nerve and other body tissues employing an implantable unpowered antenna which receives radiofrequency or microwave energy from an external generator. The benefits of atrial pacing in patients at risk of sleep apnea are described in Garrigue et al. (2002) N. Engl. J. Med. 346:404-412. Other pertinent patents include U.S. Pat. Nos. 6,289,237; 6,099,476; 5,281,219; 6,021,352; and 6,198,970.

### BRIEF SUMMARY OF THE INVENTION

The present invention provides methods and systems for treating sleep apnea, including patients suffering from or at risk of central and/or obstructive sleep apnea. The patients will have an implanted stimulator, and the methods of the present invention comprise generating a signal externally to the patient when an apnea condition exists or is likely to exist. The externally generated signal causes the stimulator to stimulate a muscle or nerve to alleviate the apnea. In some cases, the generator will be able to detect an onset of an apnea condition and to selectively generate the signal at that time. In other cases, the generator can be left on at all times while the patient is asleep in order to stimulate the target muscle or nerve in a desired pattern for the duration or preselected portions of the sleep.

Detection of the apnea condition may be performed in a variety of ways. For example, the apnea condition may be sensed externally, usually using a sensor present in or connected to the generator. Audiodetectors, motion detectors, and the like, can be used to externally detect a symptom of sleep apnea, such as breathing cessation, snoring, or the like. Alternatively, the symptom of sleep apnea may be detected internally. As well described in the patent and medical literature, internally implanted sensors can detect breathing cessation, minute ventilation diaphragm contraction, blood oxygen (P₀₂), blood carbon dioxide (P_{C02}), EEG patterns, EMG patterns, and other measurable symptoms of sleep apnea. The systems of the present invention will provide for transmission or broadcast of an alert back to the generator whenever such a condition is sensed internally.

As mentioned above, the implanted stimulator will preferably be disenabled when the patient is not sleeping. Most simply, this can be achieved by turning off the generator which produces the external signal. In cases where the generator is kept on a bedside table or other immobile position near the patient's bed, movement of the patient away from or outside of a given operational range of the generator will also disenable the stimulator. In other instances, however, the patient may wear the generator externally while sleeping. In those cases, the generator may simply be removed and turned off when not in use. In some instances, it might be desirable to automatically turn off the generator when it is removed from the patient. Additionally, it would be possible to configure the implanted stimulator to sense whether the patient is generally vertical or generally horizontal. When the patient is generally horizontal, the implanted stimulator could be enabled to receive signals. When the patient is vertical or partially vertical, however, the implanted stimulator could be disenabled so that it would be incapable of producing the desired stimulation.

As a further alternative, the patient could have implanted or externally worn sensors which are not part of the implanted stimulator. The external generator could be programmed to receive signals from the separate sensor(s), typically by powering and/or interrogating the sensors, to determine whether the patient is sleeping and/or whether a sleep apnea condition exists. Thus, the generator could determine both whether the patient or asleep and, if so, whether an apnea condition exists. For example, surface (adhesive-backed) electrodes and/or subcutaneously implanted electrodes could be provided on the scalp or elsewhere to monitor EEG waves remotely using known remote sensing systems, as described previously. The generator could monitor the heart rate and EEG wave of the patient, process those monitored signals, and initiate and adjust pacing accordingly. In particular, the EEG waves could predict the onset of an apnea event and initiate treatment through the implanted stimulator as appropriate for that point in the sleep cycle. Thus, apnea events could be prevented entirely in at least some instances.

In the preferred methods of the present invention, the generator will produce and broadcast a signal which provides both the power and the information necessary to produce the stimulatory output from the stimulator. Most simply, the generator could have an antenna which is tuned to a receiving antenna in the stimulator. The magnitude and duration of broadcast signal would then determine the magnitude and duration of stimulatory signal produced by the stimulator. In other instances, however, it may be desirable to encode the signal which is being broadcast and to provide decoding circuitry within the stimulator. In still other instances, it might be desirable to provide both a powering signal and a separate control signal which together provide the power and information necessary for the stimulator to generate a stimulatory output.

Alternatively, although generally less desirable, the implanted stimulator may include its own power source. The power source may be “permanent” in the form of a battery. More usually, however, the power source would be rechargeable, preferably being rechargeable using broadcast radiofrequency or microwave energy. In such instances, the generator would also produce a low power triggering signal to provide the necessary information for the generation of the desired stimulatory output.

In a presently preferred embodiment, the methods of the present invention will comprise positioning the generator near the patient while the patient sleeps. For example, a generator box which includes all circuitry necessary for producing and broadcasting the stimulatory signal may be placed next to the patient's bed, typically on a table or other surface near the bed. Less preferably, the external generator could be worn by the patient during sleep.

Optionally, the signal broadcast by the generator may be picked up and retransmitted before it reaches the implanted stimulator. In such instances, it may be desirable for the patient to wear an antenna or other passively powered component for both receiving the broadcast signal and retransmitting the signal to the implanted stimulator. Optionally, the antenna for rebroadcasting could include its own power source to boost the retransmitted signal before it is sent.

In another aspect, methods according to the present invention comprise implanting a stimulator which is adapted to receive an externally generated signal and produce a stimulatory pulse in response to the signal. The generator would be electrically connected to a muscle or nerve so that the stimulatory pulse will stimulate the muscle or nerve to alleviate the apnea.

Systems according to the present invention comprise an implantable stimulator and an external generator. The implantable stimulator will include an electrode for connection to a muscle or nerve. The external generator will have the circuitry necessary for broadcasting a signal which is received by the implantable stimulator to produce a stimulatory output to stimulate the muscle or nerve to alleviate the sleep apnea. The muscle may be the heart, diaphragm, hypoglossal muscle, or the like. The nerve may be the phrenic nerve, vagus nerve, or any other nerve which produces a vagal response or induces breathing.

The implantable stimulator preferably comprises decoding circuitry, and the external generator preferably comprises encoding circuitry. In this way, the signal broadcast from the generator to the implantable stimulator can be encoded so that the stimulator responds selectively only to the properly encoded signals from the generator. This will reduce or eliminate the risk of inadvertently powering or activating the stimulator from extraneous radio and electrical signals. The implantable stimulator will typically include an antenna which is tuned to receive the signal from the generator, and the stimulator is preferably adapted to be powered entirely by the broadcast signal to produce the stimulatory energy delivered to the muscle or nerve.

The system may further comprise a retransmission or booster antenna intended to receive the broadcast signal from the generator and to retransmit the signal to the stimulator. Optionally, the device may be an external antenna which is adapted to be worn by the patient. In a preferred embodiment, the external antenna would be unpowered, but in other instances, it may include a power source to provide power for boosting the signal prior to retransmission.

Further optionally, the external generator may include a detector for sensing a pattern indicating an impending apnea event, a certain stage of sleep, or a sleep apnea condition. The generator will preferably be configured to initiate broadcasting only when the stage of sleep, indication of an apnea event, or the sleep apnea condition is sensed. Usually, the detector will further be able to sense when the sleep apnea condition has ceased. The generator may then terminate the signal broadcast until a subsequent apnea condition is detected.

### BRIEF DESCRIPTION OF THE DRAWINGS

FIG. 1 illustrates a sleep apnea treatment system according to the present invention including a tabletop generator and an implantable stimulator.

FIG. 2 illustrates an exemplary implantable stimulator according to the present invention.

FIG. 3 is a schematic representation of the components of the system of the present invention.

### DETAILED DESCRIPTION OF THE INVENTION

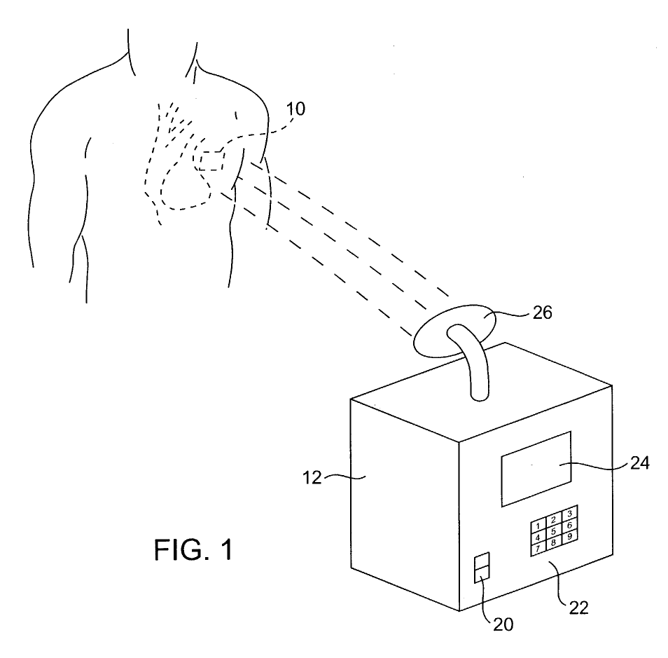

Referring to FIG. 1, a system according to the present invention comprises an implantable stimulator 10 and an external generator 12. The implantable stimulator 10 will be located near a target muscle or nerve which is desired to be stimulated in order to treat obstructive sleep apnea. Suitable muscle and nerve target sites are as follows. Nerves include the hypoglossal nerve, the twelfth cranial nerve, the phrenic nerve, the vagal nerve, carotid body baroreceptors, carotid body chemo receptors, and the like. Muscles include the hypoglossal muscles, the intercostal muscles, the heart, the diaphragm, and the like. Particularly preferred is stimulation of the right atrium of the heart in a manner analogous to cardiac pacing of the right atrium.

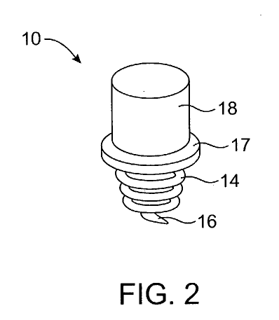

An exemplary implantable stimulator 10 is shown in detail in FIG. 2. The stimulator 10 is intended for implantation within muscle, such as heart tissue, and comprises a helical electrode 14 having a sharpened tip 16. A cylindrical capsule 18 is connected to the electrode 14 and includes an antenna and optionally circuitry for decoding signals received by the antenna, processing and forming the stimulatory output from the received signal, and the like. In the case of cardiac pacing, circuitry will typically provide a desired wave form for pacing.

Optionally, a reservoir 17 may be provided as part of the stimulator 10. The reservoir 17 could carry steroids, other anti-inflammatory drugs, or a variety of other substances, which could be continuously or selectively delivered to the muscle or nerve in which the stimulator is implanted. The drug of other substance could be released directly from the reservoir 17 using a variety of well-known controlled release techniques, or alternatively could be eluted through the electrode 14 which, for example, could be formed as a hollow perforate or porous structure connected to the reservoir 17.

To prevent extraneous signals from triggering the stimulator 10, generator 12 may produce a series of two, three or more select signals to trigger the stimulator, with the appropriate circuitry for detecting those signals provided within the capsule 18. For example, a digital “key” signal may be transmitted by the generator together with or as part of the pacing signal. Circuitry within the stimulator would detect the key signal to enable operation of the stimulator. In the absence of the key signal, the stimulator would not function. In some instances, several digital key signals could be transmitted simultaneously. One key signal could tell the stimulator to fire, while another key signal would designate which wave form would be utilized. In some instances it may be desirable to employ one pacing wave form during one stage of sleep and one or more other pacing wave forms during other stages of sleep.

The generator 12 will include an off/on switch 20, a touch pad 22 for inputting control information, an LCD or other display for displaying information, and a broadcast antenna 26 intended to broadcast the stimulatory signal to the implanted stimulator 10.

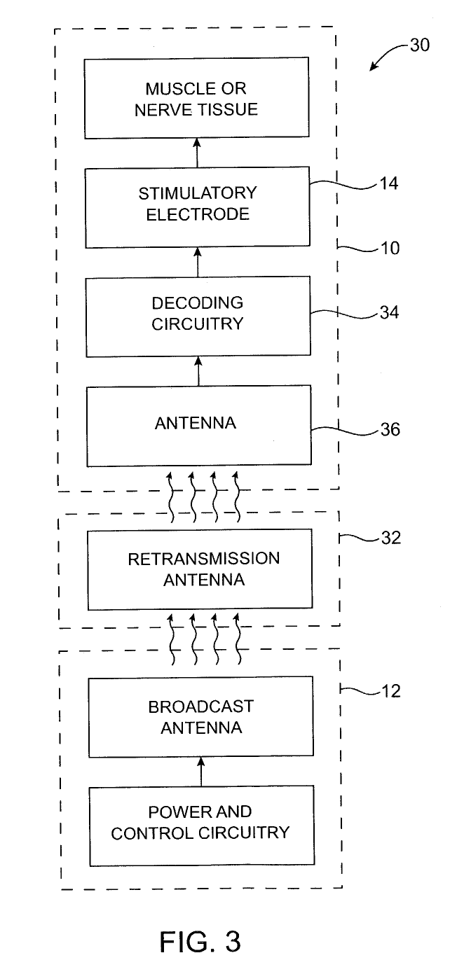

Referring now to FIG. 3, in the case of cardiac stimulation, the implantable stimulator 10 may be introduced by minimally invasive and/or endovascular techniques. Particularly, for atrial pacing, the stimulator 10 could be introduced through a direct percutaneous introduction through the chest wall. To access the left atrium, a needle-type introducer carrying the stimulator directly between the ribs could be advanced under fluoroscopic guidance. Alternatively, the introducer could be advanced between the upper right ribs and pass beneath the sternum.

Generally, however, endovascular techniques will be preferred. To place the stimulator 10 in the right atrium via an endovascular technique, a catheter may be used to access the femoral, radial, brachial, or subclavian veins over a guidewire in a conventional manner to the right atrium. The implantable stimulator 10 would be advanced through or carried by the delivery catheter. In the case of the femoral vein, the catheter would traverse the inferior vena cava into the right atrium. For the subclavian, radial, or brachial veins, the catheter would pass through the superior vena cava and into the right atrium. A variety of suitable steerable delivery catheters exist for implanting the stimulator 10, typically by engaging the sharpened tip 14 against the endocardium and rotating the stimulator so that the helical coil electrode 16 is implanted.

Referring now to FIG. 3, systems 30 according to the present invention comprise the implantable stimulator 10, the external generator 12, and optionally a retransmission antenna 32. The implantable stimulator 10 will include the stimulatory electrode 14 which is implanted or otherwise electrically coupled to the target muscle or nerve tissue. Optionally, decoding circuitry 34 receives the broadcast or retransmitted signal from antenna 36. The decoding circuitry will pass the signal from the antenna to the stimulatory electrode if and only if the signal from the generator 12 has been properly encoded. Usually, additional circuitry will be provided for conditioning and forming the signal in the desired wave pattern to achieve the needed muscle or nerve stimulation.

The external generator 12 will include power and control circuitry for generating a signal which is broadcast via the broadcast antenna. Optionally, the generator will also include circuitry for externally detecting or sensing when a patient is displaying a sleep apnea condition, such as breathing cessation, snoring, or the like.

The retransmission antenna is optionally provided to receive and boost the signal from the broadcast antenna and pass the boosted signal along to the receiving antenna 36 in implantable stimulator 10.

What is claimed is:

1. A method for treating sleep apnea in a patient having an implanted stimulator, said method comprising:
generating a signal externally to the patient when an apnea condition exists or is likely to exist;
wherein said signal causes the stimulator to stimulate a muscle or nerve to alleviate the apnea.

2. A method as in claim 1, further comprising detecting the apnea condition, wherein the signal is generated when apnea is detected.

3. A method as in claim 2, wherein detecting the apnea condition comprises externally sensing a symptom of apnea.

4. A method as in claim 3, wherein the symptom is selected from the group consisting of breathing cessation and snoring.

5. A method as in claim 2, wherein detecting comprises internally sensing a symptom of apnea.

6. A method as in claim 5, wherein the symptom is selected from the group consisting of breathing cessation, minute ventilation, blood oxygen, blood carbon dioxide, diaphragm contraction, EEG waveform, and EMG waveform.

7. A method as in claim 1, wherein the implanted stimulator is disenabled when the patient is not sleeping.

8. A method as in claim 1, wherein the implanted stimulator is disenabled when the patient is upright.

9. A method as in claim 1, wherein the signal from the generator is encoded and the stimulator stimulates the muscle or nerve only in response to the encoded signal.

10. A method as in claim 1, wherein the signal both triggers and powers the stimulator to stimulate the muscle or nerve.

11. A method as in claim 10, wherein generating comprises transmitting power which is received by an implanted antenna and which directly delivers energy of a magnitude and duration which stimulates the muscle or nerve.

12. A method as in claim 11, wherein the transmitted power is encoded so that only the transmitted power can cause the stimulator to deliver energy to the muscle or nerve.

13. A method as in claim 1, further comprising transmitting power from an external source to a power receiver and accumulator implanted in the patient, wherein the accumulator is connected to deliver power to the stimulator.

14. A method as in claim 13, wherein generating a signal comprises transmitting the signal to the stimulator separately from transmitting power to the power receiver and accumulator.

15. A method as in claim 1, wherein generating comprises positioning a generator near the patient while the patient sleeps.

16. A method as in claim 15, wherein positioning comprises placing the generator on a surface near the patient.

17. A method as in claim 15, wherein positioning comprises securing the generator externally to the patient.

18. A method as in claim 15, further comprising retransmitting the signal to increase the power to the stimulator.

19. A method as in claim 18, wherein retransmitting comprises the patient wearing an external antenna which receives the generated signal and retransmits the signal to the stimulator.

20. A method as in claim 20, wherein the external antenna is connected to a power source to boost the retransmitted signal.

21. A method as in claim 20, wherein the external antenna is unpowered.

23. A method as in claim 22, wherein the signal paces the right atrium

24. A method for treating sleep apnea in a patient, said method comprising:
implanting a stimulator which is adapted to receive an externally generated signal and to produce a stimulatory pulse in response to the signal; and
electrically connecting the implanted stimulator to a muscle or nerve so that the stimulatory pulse will stimulate the muscle or nerve to alleviate the apnea.

25. A system for treating sleep apnea in a patient, said system comprising:
an implantable stimulator having an electrode for connection to a muscle or a nerve; and
an external generator which broadcasts a signal which is received by the implantable stimulator to produce energy to stimulate a muscle or nerve to alleviate the sleep apnea.

26. A system as in claim 25, wherein the implantable stimulator comprises decoding circuitry and the external generator comprises encoding circuitry, wherein the stimulator responds selectively to signals from the generator.

27. A system as in claim 25, wherein the generator broadcasts a radiofrequency or microwave signal and wherein the implantable stimulator includes an antenna which is tuned to receive the signal from the generator.

28. A system as in claim 25, wherein the implantable stimulator is adapted to be powered entirely by the broadcast signal to produce the stimulatory energy delivered to the muscle or nerve.

29. A system as in claim 25, further comprising an external antenna, to be worn by the patient, wherein the external antenna receives the broadcast signal from the generator and retransmits the signal to the implantable stimulator.

30. A system as in claim 29, wherein the external antenna includes a power source.

31. A system as in claim 29, wherein the external antenna is unpowered.

32. A system as in claim 25, wherein the external generator includes a detector for sensing a sleep apnea condition in the patient and initiating signal broadcasting when the condition is sensed.

33. A system as in claim 32, wherein the detector senses when the sleep apnea condition has ceased and terminates signal broadcasting when the termination is sensed.

34. A system as in claim 1, wherein the implantable stimulator is adapted for implementation in or on a heart.

35. A system as in claim 34 wherein the stimulator is adapted for pacing within a right atrium.
